(19) United States
(12) Patent Application Publication
(10) Pub. No.: US 2019/0148015 A1
(43) Pub. Date: May 16, 2019

(54) MEDICAL INFORMATION PROCESSING DEVICE AND PROGRAM

(71) Applicant: KONICA MINOLTA, INC., Tokyo (JP)

(72) Inventors: Hitoshi FUTAMURA, Tokyo (JP); Satoshi KASAI, Tokyo (JP)

(73) Assignee: KONICA MINOLTA, INC., Tokyo (JP)

(21) Appl. No.: 16/191,137

(22) Filed: Nov. 14, 2018

(30) Foreign Application Priority Data
Nov. 16, 2017 (JP) 2017-220537

(51) Int. Cl. G16H 50/20; G16H 30/40

(52) U.S. Cl. CPC G16H 50/20; G16H 30/40

(57) ABSTRACT

A medical information processing device includes: a hardware processor that: acquires data regarding a human body; accumulates acquired data acquired by the hardware processor; analyzes accumulated data accumulated by the hardware processor, autonomously finds a correspondence pattern between the accumulated data and at least timing at which prescription is performed and a content out of the accumulated data and generates an identifier used for inference; infers a prescription content including at least the timing at which the prescription should be performed using the identifier generated by the hardware processor and the acquired data; and outputs the prescription content inferred by the hardware processor.

The entire disclosure of Japanese patent Application No. 2017-220537, filed on Nov. 16, 2017, is incorporated herein by reference in its entirety.

### BACKGROUND

### Technological Field

The present invention relates to a medical information processing device and a program.

### Description of the Related Art

As a technology of assisting determining prescription to a subject (patient and the like) by a medical worker and the like, for example, technologies disclosed in JP 5744631 B2, JP 11-047096 A, and JP 07-175404 A are proposed.

A diagnosis assisting device disclosed in JP 5744631 B2 calculates a characteristic value by analyzing a medical image, infers a diagnostic name and acquires reliability thereof from the characteristic value, and presents a medical practice which should be performed next held in advance in a case where the characteristic value does not satisfy a predetermined standard.

In addition, a health management system disclosed in JP 11-047096 A is to infer any one of prediction of illness or risk, proper exercise prescription, proper diet prescription from personal vital data and lifestyle data.

In addition, a health guidance system disclosed in JP 07-175404 A acquires intake calories from meals, a nutrient intake amount, consumption calories of various exercises from medical interview data, dietary data, and exercise activity data (referred to as living situation data), and creates an optimal lifestyle habit improvement menu in dietary life and exercise life based on the accumulated living situation data.

In recent years, a steep rise in medical cost due to aging becomes a social problem. In contrast, medical financial resources and medical resources are limited, and it is required to conduct medical care or therapy for a whole without excess or insufficiency.

However, the technologies disclosed in JP 5744631 B2, JP 11-047096 A, and JP 07-175404 A do not perform learning in which accumulated data and prescription are associated with each other, but determine the prescription by checking an analysis result of the data against an index determined in advance such as a guideline. Therefore, as a result of the analysis, timing and a type of the prescription are uniformly set for subjects determined to be in similar states. However, since there are various detailed states of the subjects, there might be ones with excessive prescription and ones with insufficient prescription. If the prescription is excessive, there is a wasted medical cost correspondingly, and if the prescription is insufficient, it means that a quality of medical care is not sufficient.

In order to solve such a situation, a manner of deriving prescription is adjusted. However, if it is adjusted to reduce ones with excessive prescription, the number of ones with insufficient prescription increases, and on the other hand, if it is adjusted to reduce ones with the insufficient prescription, the number of ones with the excessive prescription increases.

That is, with the conventional technology of assisting determining the prescription, individual optimization of the prescription is not sufficient, so that there is a trade-off between an increase in medical cost and deterioration in medical quality.

### SUMMARY

The present invention is achieved in view of the above-described problems, and an object thereof is to reduce excess or insufficiency of the prescription in a device of presenting prescription to a subject based on data acquired from the subject.

To achieve the abovementioned object, according to an aspect of the present invention, a medical information processing device reflecting one aspect of the present invention comprises: a data acquirer that acquires data regarding a human body; a data accumulator that accumulates acquired data acquired by the data acquirer, a prescription learner that analyzes accumulated data accumulated by the data accumulator, autonomously finds a correspondence pattern between the accumulated data and at least timing at which prescription is performed and a content out of the accumulated data and generates an identifier used for inference; a prescription inference part that infers a prescription content including at least the timing at which the prescription should be performed using the identifier generated by the prescription learner and the acquired data; and an output part that outputs the prescription content inferred by the prescription inference part.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a block diagram illustrating a configuration of a medical information processing device according to an embodiment of the present invention;

FIG. 2 is a flowchart illustrating a learning process executed by the medical information processing device in FIG. 1;

FIG. 3 is a flowchart illustrating a prescription presenting process executed by the medical information processing device in FIG. 1;

FIG. 4 is a setting example of an inference processing condition in the medical information processing device in FIG. 1;

FIG. 5 is a setting example of an input condition of data in the medical information processing device in FIG. 1;

FIG. 6 is a conceptional diagram illustrating a prescription presenting process executed by the medical information processing device in FIG. 1;

FIGS. 7A and 7B are examples of presenting prescription by the medical information processing device in FIG. 1; and

FIGS. 8A to 8C are input examples of a result in the medical information processing device in FIG. 1.

### DETAILED DESCRIPTION OF EMBODIMENTS

Hereinafter, one or more embodiments of the present invention will be described in detail with reference to the drawings. However, the scope of the invention is not limited to the illustrated example.

[Configuration of Medical Information Processing Device]

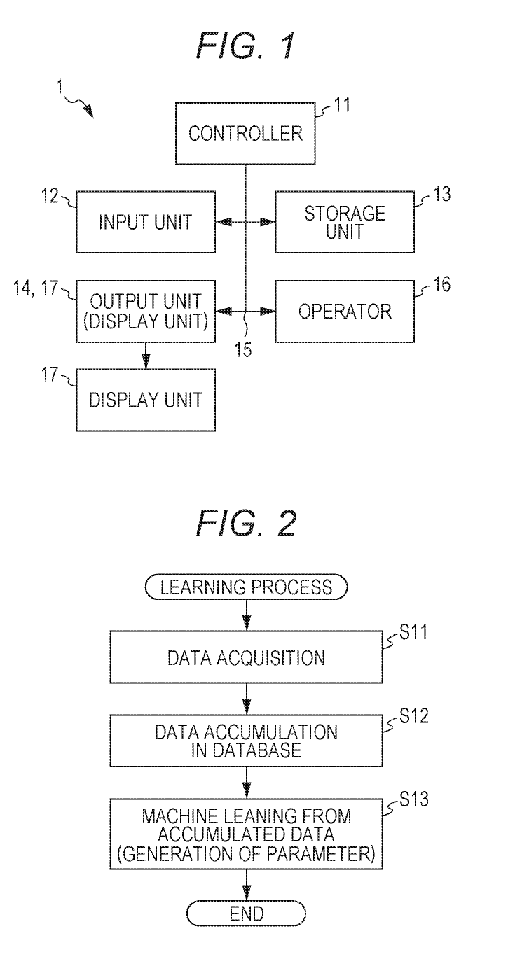

First, a configuration of a medical information processing device 1 according to this embodiment is described. FIG. 1 is a block diagram illustrating the configuration of the medical information processing device 1.

The medical information processing device 1 acquires and accumulates input or generated data, a method of acquiring the same, and an occurred event, learns correspondence between an acquired data group and timing at which next prescription is performed and a type thereof, and infers timing at which next prescription is performed and a type thereof for preventing a future event based on a learning result.

The medical information processing device 1 may be used in various scenes such as medical checkup, outpatient care, emergency, hospitalization (ward), rehabilitation, nursing care, and medical home care; this is especially useful in the medical checkup.

The medical information processing device 1 is configured as a PC, a portable terminal, or a dedicated device, and is provided with a controller 11, an input unit 12, a storage unit 13, an output unit 14 and the like as illustrated in FIG. 1. The units 11 to 14 are connected to one another by a bus 15.

The medical information processing device 1 of this embodiment is also provided with an operator 16 in addition to them.

The controller 11 is configured to comprehensively control operation of each unit of the medical information processing device 1 by a CPU, RAM and the like. Specifically, this reads various programs stored in the storage unit 13 and develops the same on the RAM, and executes various processes according to the programs.

The input unit 12 inputs various data regarding a human body from an external device (various medical devices (plain X-ray, MMG, US, CT, MRI and the like), a picture archiving and communications system (PACS), an electronic health record device, and other systems).

The input unit 12 is preferably formed of a network interface and the like so as to receive the data from the external device connected by wire or by radio through a communication network such as a local area network (LAN), a wide area network (WAN), and the Internet; however, it is also possible to form the same of a port and the like in which a USB memory, an SD card and the like may be inserted.

The storage unit 13 is formed of a hard disk drive (HDD), a semiconductor memory and the like and stores the program for executing various processes including machine learning to be described later, a parameter required for executing the program, a file and the like.

In the storage unit 13, a database for storing externally acquired data and data generated by inference by the device itself is constructed.

Specific examples of the data which may be accumulated in the database include, for example, medical images (plain X-ray, US, CT, MRI, PET, RI, endoscope, IVR, pathology and the like), photographs (body surface (face, skin and the like), bodily wastes (excrement and vomit), meal, room situation and the like), vital data (pulse oximetry and electrocardiogram), a blood examination, an urinary examination, a medical interview, records (nursing record, diary and the like) and the like.

Note that the database may also be provided in storage means other than the storage unit 13.

In a state before the medical information processing device 1 is used in a medical institution and the like, an initial parameter is stored in the storage unit 13.

Thereafter, the medical information processing device 1 operates, accumulation of the data in the database progresses, and the parameter is updated each time a learning process to be described later is executed.

Note that a parameter which another device learns in advance may be used as the initial parameter.

The output unit 14 outputs information processed by the medical information processing device 1.

The output unit 14 may be, for example, a display device for displaying a processing result, a printer itself for printing the processing result, or a connector for connecting to them. This may also be a network interface for communicating with another system and the like, or a port for various media such as a USB memory.

Note that the input unit 12 may also serve as the output unit 14.

The operator 16 is configured so as to be operable by a user with a keyboard provided with various keys, a pointing device such as a mouse, a touch panel attached to the display device or the like, and outputs an operation signal input according to key operation on the keyboard, mouse operation, or a position of touch operation on the touch panel to the controller 11.

Note that it is also possible that the portable terminal is connected by wire or by radio, and a liquid crystal display panel of this portable terminal is used as the output unit 14 and the touch panel and the button is used as the operator 16.

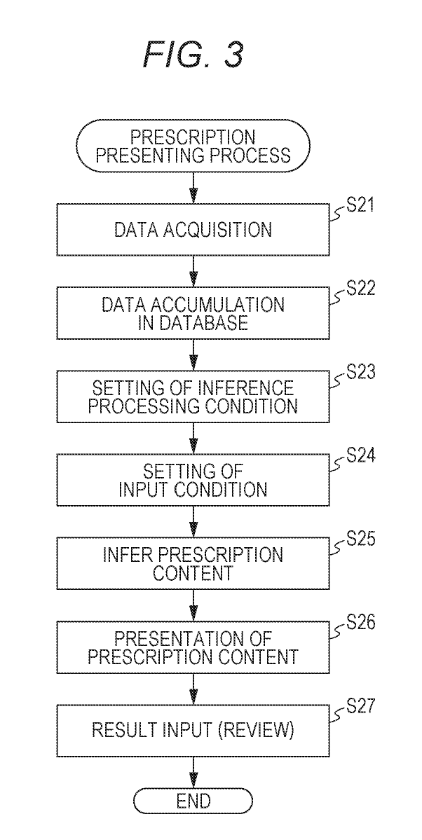

Next, operation of the medical information processing device 1 is specifically described. FIG. 2 is a flowchart illustrating the learning process executed by the medical information processing device 1, and FIG. 3 is a flowchart illustrating a prescription presenting process executed by the medical information processing device 1.

The controller 11 of the medical information processing device 1 configured as described above executes the learning process illustrated in the flowchart in FIG. 2 by an external data input, operation of instructing the operator 16 to start processing and the like as a trigger, for example.

This learning process is performed at the time of development/manufacture of the medical information processing device 1, between prescription presenting processes to be described later and the like.

In the learning process, first, the data regarding the human body is acquired (step S11), and the acquired data is accumulated (step S12). Specifically, at least any one of a plurality of types of input data input from the input unit 12, the operator 16 and the like and information regarding the occurred event is accumulated in the database of the storage unit 13.

Herein, the “event” is intended to mean deviation of a state of a subject from a current state. Specifically, this includes onset from a presymptomatic state, recurrence of illness from a completely healed state or remission, a turn to a severe state from a state of emergency transport and the like.

Note that, a case of changing to an improved state, that is, a case of turning to the completely healed state or the remission from the onset state, a resting state from any defect state and the like are also included in the “event”.

By executing this process, the controller 11 and the input unit 12 form a data acquirer in the present invention, and the controller 11 and the storage unit 13 form a data accumulator in the present invention.

The data which may be acquired by this process includes, for example, image data and text data. It is possible to input either of them, but it is preferable that both of them are included.

The image data includes a plain X-ray image, a US image, a CT image, an MRI image, a pathological image and the like.

In contrast, the text data includes measured value data, subjective data and the like.

The measured value data includes, for example, a body height and a body weight, the blood examination, the urinary examination, a pedometer, the pulse oximeter, the electrocardiogram and the like.

The subjective data includes, for example, a content of description in a medical interview sheet, a recorded content recorded by a medical worker, a diary written by a patient and the like.

Note that, in a first learning process performed at the time of development/manufacture, a large amount of such data is collected from medical institutions and the like and input to the input unit 12 to construct a database.

In learning processes executed for a second time and thereafter, the prescription presenting process to be described later is performed before this process, and the data is acquired and accumulated in the prescription presenting process, so that it is possible that processes at steps S11 and S12 are not performed.

In order to collect and accumulate various types of information which might be the above-described data in an integrated manner, there is a case that the input unit 12 alone is insufficient. For example, this is a case in which data of information required for learning is recorded and accumulated in an external electronic health record device, but in communication between the electronic health record device and the medical information processing device 1, there is no protocol for directly exchanging an input item as digital data.

In this case, it is also possible to provide a mechanism of acquiring the text and image recorded in the electronic health record device by directly inputting image information output on a display screen of the electronic health record device to the medical information processing device 1 (performing image capturing) and learning a user's operation pattern from the input image.

Specifically, a screen layout and a change pattern are learned so that timing at which the data appearing on the screen such as a confirmation button and a transmission button is confirmed may be detected, and at the time of detection, predetermined data is acquired from a predetermined region by using immediately preceding image data. Herein, in a case where it is wanted to convert to the text data, general optical character recognition (OCR) software and the like may be used.

In this manner, it becomes possible to acquire learning data even in a case where the digital data cannot be directly exchanged, and it is possible to omit effort of inputting the data once input by another system such as the electronic health record device again to the information processing device 1 to execute an inference process.

After accumulating the data, the machine learning is performed using the accumulated data (step S13). Specifically, the accumulated data accumulated in the database is analyzed, a correspondence pattern between the accumulated data and at least timing at which the prescription is performed and the content thereof is autonomously found out of the accumulated data, and a parameter (identifier) used for the inference is generated. That is, the controller 11 and the storage unit 13 form a prescription learner in the present invention.

Note that it is preferable to perform the machine learning using data groups of various combinations to generate a plurality of types of parameters.

As the machine learning, it is also possible to perform deep learning which is a type thereof. That is, the learning is performed using a neural network having a multilayer structure.

In conventional machine learning, it is necessary that a developer define and design a feature to be identified, but it is said that the deep learning also has a feature extracting function, and it is expected to improve accuracy by generating a feature independent of human viewpoints.

The controller 11 of the medical information processing device 1 executes the prescription presenting process illustrated in the flowchart in FIG. 3 by the external data input, the operation of instructing the operator 16 to start processing and the like as a trigger, for example.

In the prescription presenting process, first, the processes the same as those at steps S11 and S12 in the above-described learning process, that is, acquisition of the data regarding the human body (step S21) and accumulation of the acquired data (step S22) are performed.

After accumulating the data, an inference processing condition is set (step S23). Specifically, data to be used for inference is selected out of a plurality of types of acquired data.

As a manner of selection, for example, there is a method of narrowing the data by a range, a type, an accumulation period and the like.

The range is, for example, data of all people accumulated, data of only the same person, data of persons having the same attribute and the like. Herein, “the same attribute” includes, for example, the same generation (forties, fifties, 45 to 65 years old and the like), the same sex, the same examination result range (for example, BMI≥25) and the like.

The type is, for example, a medical department, the image data, the measured value data, the subjective data and the like.

The accumulation period is, for example, data of an entire period from the start of accumulation, data of the latest several years (for example, five years, ten years and the like) and the like.

Note that, as for the period, it is also possible to provide a numerical value input field E so that an arbitrary specified period (for example, years 2012 to 2016 and the like) may be specified.

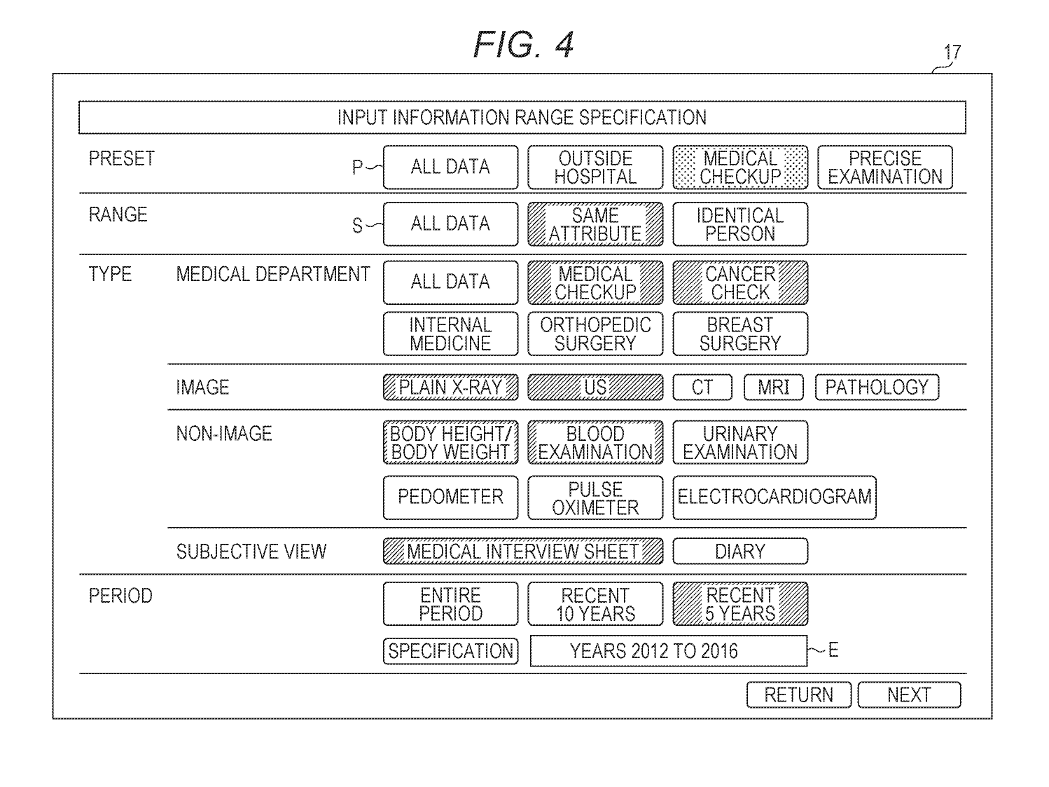

In a case where the output unit 14 is formed of the display device or the display device is connected to the output unit 14, it is also possible to display a range specification screen in which options (range, type, period and the like) for each item are listed as illustrated in FIG. 4 on the display unit 17 of the display device and allow the user to select while watching the same.

Also, if a plurality of combinations obtained by selecting the data from each item such that there is specific directivity is made presets P (for example, all data, out of hospital, medical checkup, precise examination and the like) and the preset is selected, it becomes not necessary to select the option for each item.

Especially, if the operator 16 is attached to the display unit 17 of the display device as the touch panel, it is possible to set only by touching display of the options and presets displayed on the display unit 17, so that the inference processing condition may be more easily set.

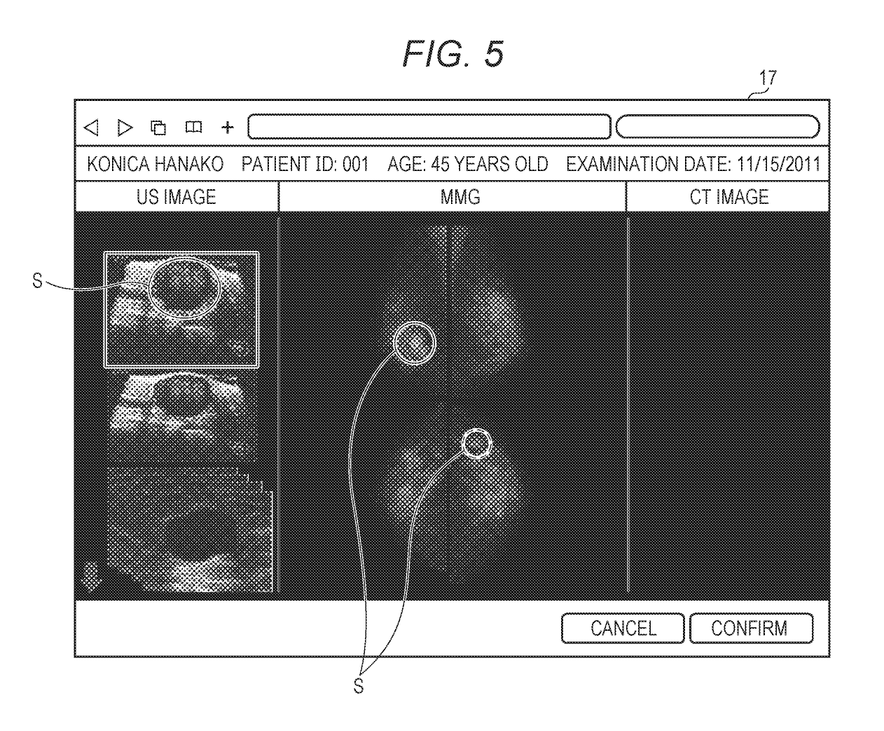

After setting the inference processing condition, a data input condition (especially, that acquired this time) is set (step S24). In this process, for example, in a case of inputting the image data, an interpretation report, a feature and the like of the input image data are input. Specifically, as illustrated in FIG. 5, for example, an image based on the image data is displayed on the display device, and a region of interest is indicated by a figure S surrounding the same. Note that, the figure S to indicate is exemplified by an ellipse in the drawing, but a shape thereof may be arbitrary such as a rectangle, a circle, a polygon, a free curve and the like.

Note that it is preferable that at least two types of image data acquired from different modalities are included in the image data.

It is also possible to apply region information already set by another device to the region of interest. For example, a plurality of figures S may be displayed in an initial state, and an arbitrary figure S selected by the user may be set as the region of interest.

Also, in a case where data is missing at this stage, the user may input the same. That is, an input based on the operation performed on the operator 16 may be used as the acquired data.

As data input in place of the missing data, it is possible to select a standard value (normal value), a statistical value and the like. The statistical value is, for example, a value calculated from values aggregated in specific generation or sex.

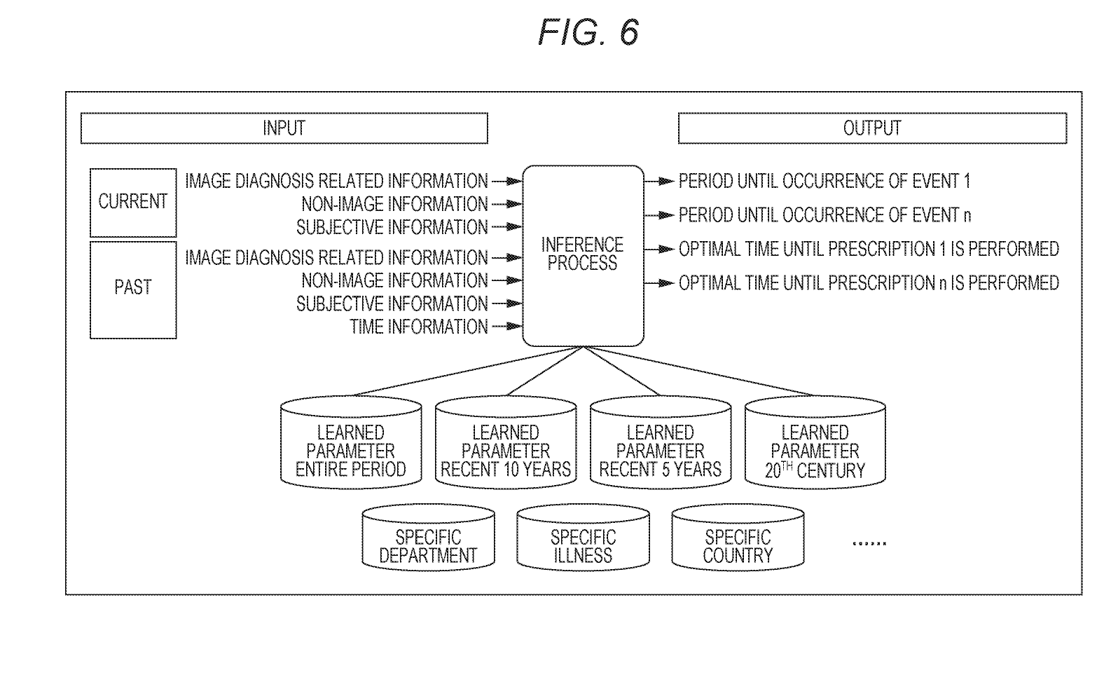

After setting the input condition, a prescription content is inferred (step S25). Specifically, as illustrated in FIG. 6, the prescription content including at least the timing at which the prescription should be performed is inferred using the parameter generated by executing the above-described leaning process and the acquired data. Specifically, the prescription content may be acquired by applying the acquired data to the generated parameter. That is, the controller 11 forms a prescription inference part in the present invention.

Note that, it is also possible to infer the type of the prescription (including an examination condition; for example, in a case of plain X-ray examination, this is a shooting condition) together as the prescription content.

In a case where a plurality of types of parameters is stored, it is also possible to infer a plurality of types of prescription contents by using a plurality of types of parameters and acquired data.

Then, since the imaging condition is usually determined with reference to a body type of a subject being tested, a past medical record and the like regardless of doctor's preference, thinking characteristics and the like, so that there is a problem that it cannot reach individual optimum (solution of trade-off between diagnostic performance improvement and exposure dose control).

Therefore, the imaging condition may also be inferred from the medical interview, past examinations, BMI, the doctor's preference and the like.

In this manner, it is possible to realize the individual optimum to solve such trade-off.

After executing the inference process, the inferred prescription content is presented (step S26). In this embodiment, the prescription is displayed on the display unit 17 of the display device forming the output unit 14 or connected to the output unit 14 to be presented.

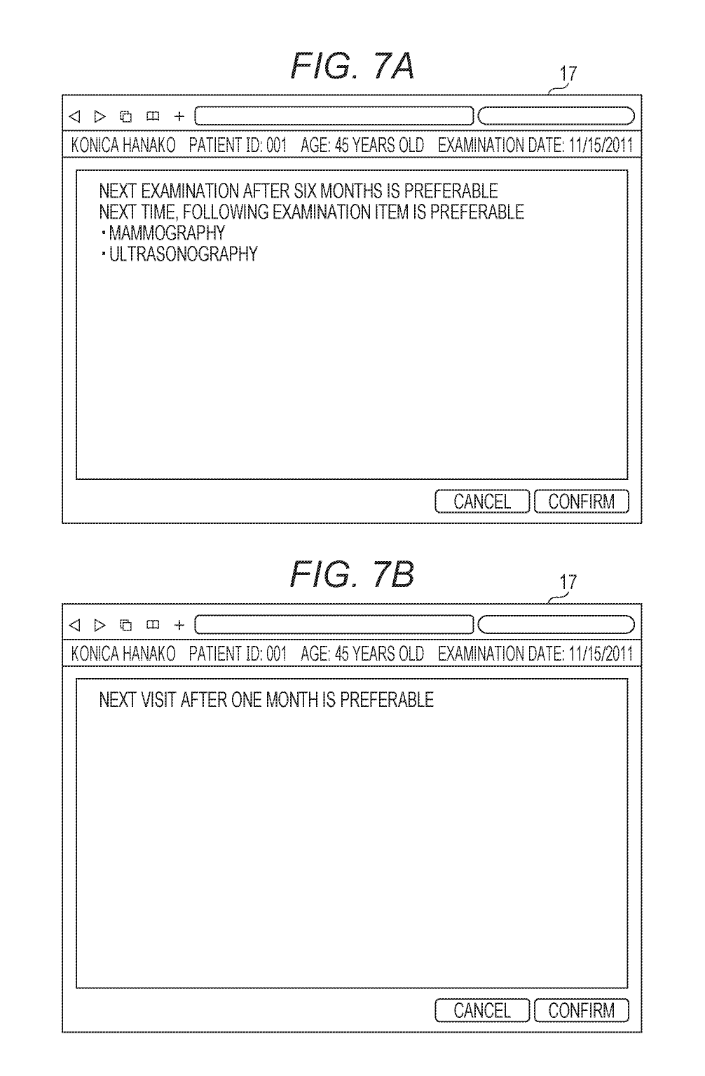

For example, as illustrated in FIGS. 7A and 7B, next examination date and visit date (for example, six months later and the like), a next examination item (for example, mammography, ultrasonography and the like) and the like are presented. Note that, although examples of presenting the prescription to a patient and an examiner of the medical checkup are illustrated in FIGS. 7A and 7B, the prescription directed to a medical worker and the like may also be presented.

By performing this process, the controller 11 and the output unit 14 (display device) serve as an output part in the present invention.

In the medical information processing device 1 of this embodiment, the following is supposed as a combination of the presentation made for the input.
A period to a next examination and a type of the examination (dosing period, medical checkup interval) are presented for an input of a radiation image, a medical interview result, and a blood examination result. Note that when the radiation image is input, the examination condition may also be presented in addition to the period until the next examination and the type of the examination.
The period until the next examination and the type of the examination are presented for an input of mammography and/or ultrasonography and/or medical interview.
Round timing and scheduled behaviors (examination, procedure and the like) are presented for an input of a nursing care report and various sensors.
The round timing is presented for an input of various vitals and examination results (in a case of use in ICU and the like).
Excised end additional cutting is presented for an input of the pathological image (in a case where the event already occurs).
Timing of a next additional examination is presented for an input of the medical checkup result.

Note that in initial medical care of breast cancer and the like, either the medical interview, palpation, a MMG examination, or a US examination, or a combination thereof is executed, and then next prescription is determined. Therefore, in a case where it is determined that findings found in the initial medical care are highly likely to be malignant, it is determined whether this is benign or malignant by conducting additional diagnosis such as highly invasive cytodiagnosis or tissue diagnosis; however, if such additional diagnosis is carried out excessively, this might increase physical, financial, and psychological burdens of the patient. On the other hand, if the additional diagnosis is excessively suppressed, cancer might be overlooked. Solution of such trade-off becomes a big problem in medical scenes.

Therefore, in addition to presenting the content of the prescription to be performed at a later date as described above, there may be a case of presenting the content of the prescription to be performed immediately later (the same date) (for example, necessity of cytodiagnosis, necessity of tissue diagnosis and the like).

Also, when presenting the prescription, information regarding the prescription content, specifically, a reason for the inference of the presented prescription content may also be presented together. In this manner, it is easy for the subject to consent as compared with a case where only the presentation result is given.

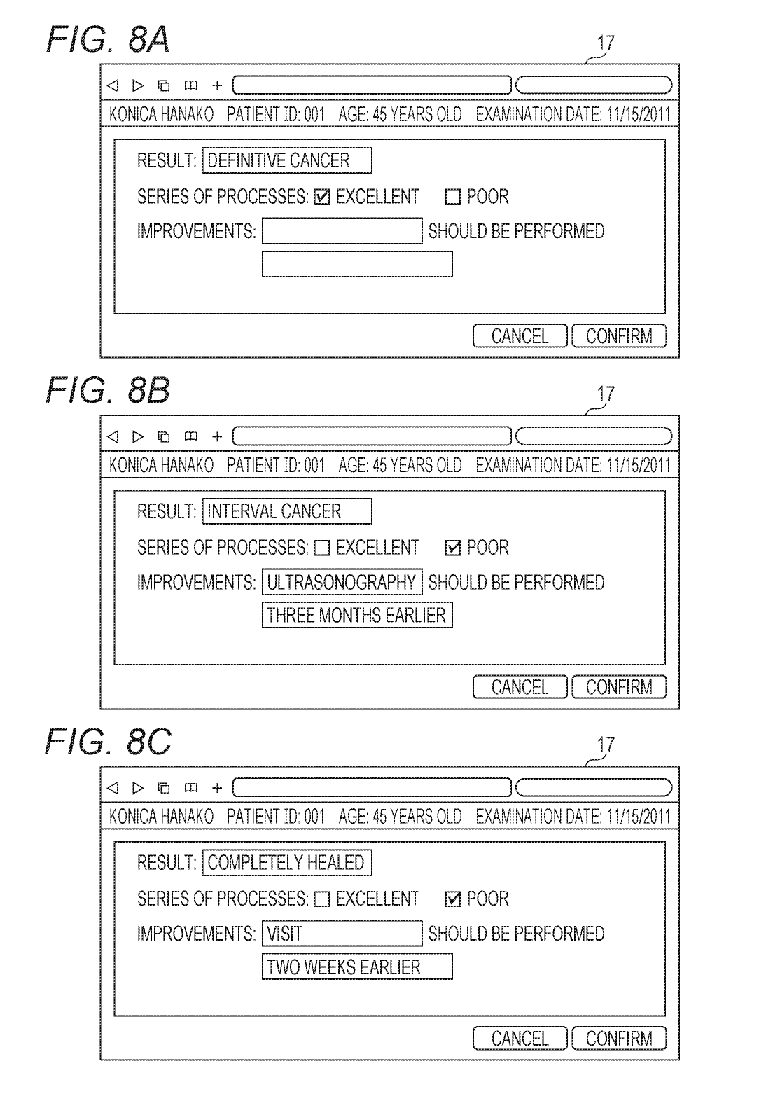

In the prescription presenting process of this embodiment, after presenting the prescription and the like, an input of a result (review) is accepted (step S27). Specifically, as illustrated in FIGS. 8A to 8C, a final result of the subject (type of illness, whether this is completely healed and the like), whether a series of procedures performed so far is excellent as a result and the like.

In a case where it is input that the result is excellent, this is accumulated as data of a case in which a series of procedures is successful, and in a case where it is input that the result is not excellent, this is accumulated in the database as data of a case in which a series procedures is not successful. As a result, it becomes possible to learn patterns of appropriate procedure.

Note that, in a case where the content that the prescriptions so far are not excellent is input, it is also possible to input and accumulate improvements (when and which type of procedure should be performed and the like) and this may be reflected to the learning.

The medical information processing device 1 of this embodiment not derives the prescription according to the information held in advance (for example, a guideline (practice guideline and standard therapy) designed based on studies of academic societies and the like) as in the conventional manner but infers the prescription content by using the parameter generated by performing the machine learning by using the accumulated information. Since the parameter is different for each subject, even if the data in a similar state is input, the prescription content is different for each subject. That is, the medical information processing device 1 of this embodiment may present an optimal prescription content taking into consideration of difference in a detailed state between the subjects.

In a case where the doctor prescribes for a target human body (patient or subject being tested) having similar characteristics, the prescription content might change depending on difference in attribute of the doctor (academic clique, specialty, and country (culture)).

Therefore, in the medical information processing device 1 according to the above-described embodiment, the learning and inference may be performed for each attribute of the doctor.

In that case, the attribute of learned data used in the inference at step S26 (for example, XX University hospital affiliate and the like) may be presented together, and further, a plurality of inference results inferred from a plurality of learning data (both a result of XX University hospital affiliate and a result of YY hospital affiliate) may be presented.

In this manner, it is expected that a degree of consent of the user increases.

Also, due to characteristics of a general hospital, not all doctors working at a certain hospital necessarily have the same attribute, and it is not realistic to strictly assign an attribute to each doctor and operate this medical information processing device 1. As a result, it is expected that the possibility of accumulating the learning data with correct attribute data is lowered, and it is highly likely that excellent inference accuracy cannot be acquired by the learning using such data.

Therefore, it is possible to calculate a degree of similarity to the accumulated data by inputting the image or information input from the user, and accumulate the input data with poor similarity as data having another attribute.

In this manner, it is possible to accumulate high-quality learning data for realizing a highly accurate inference process.

In addition, for the doctor who mainly uses the medical information processing device 1 according to the above-described embodiment, motivation to leave a medical record as correct digital data is often low because he/she is busy without time. For example, if handwritten paper health record is recorded, there is no problem in the medical care, and even if the electronic health record is used, the possibility that this is written in signals and the like discriminable only for him/her cannot be denied. There also is the possibility of misspellings and omissions in plain text input which is not multiple-choice type.

Therefore, it is possible to incorporate a function of making arrangement useful for a user in return for correct digital data providing.

For example, points are added according to a case of recording correct annotation in the image, a case of inputting definite diagnosis and the like, and incentive is set according to the points. The incentive is, for example, discounts at the time of next purchase of equipment, and extension of trial periods and increase in trial times of new products.

For determining whether the annotation is correct, it is possible to calculate a degree of similarity between the data group already accumulated and determined to be the correct annotation and the data input this time, and determine that the annotation is correct if the degree of similarity is high, or may measure an annotation recording time (from start to end) and determine that the annotation is correct when the time is long.

As a result, the doctor's motivation is improved, and the learning data as the correct digital data may be collected.

In addition, when the user feels that operability or visibility is poor when using the medical information processing device 1 according to the above-described embodiment, there is a risk that the user stops recording due to irritation. There might be a plurality of factors such as a product design defect, deterioration in user using environment, user's physical disorder and the like. If the learning data cannot be accumulated and the inference process is not used, there is disadvantage for patients and subjects to be tested.

In such a case, even if there is a function of making arrangement useful for the user and improving user's motivation to the input, this cannot function well.

Therefore, it is possible to infer a degree of irritation of the user by using record of user operation (trajectory and speed of a mouse pointer), a used function, and an application situation (response time and the like) as an input and perform a predetermined action in a case where an inference result is not excellent.

The predetermined action may be automatic contact to a customer support, recommendation to change to a screen layout used by another user with an excellent degree of irritation (not irritated), or reminder of a result to the user.

As a result, it is possible to suppress the user's irritation and make the user properly use the medical information processing device 1.

Note that, an example in which a HDD and a semiconductor memory is used as a computer readable medium of the program according to the present invention is described, but the present invention is not limited to this example.

As another computer readable medium, a non-volatile memory such as a flash memory and a portable recording medium such as a CD-ROM is applicable.

A carrier wave is also applied to the present invention as a medium for providing data of a program according to the present invention via a communication line.

According to an embodiment of the present invention, it is possible to prescribe according to a state of each subject, so that excess or insufficiency of prescription is reduced and it is possible to divide the subjects who need more prescription than general standards from the subjects who need less prescription. As a result, it is possible to satisfy both decrease in medical cost and improvement in medical quality.

Although embodiments of the present invention have been described and illustrated in detail, the disclosed embodiments are made for purposes of illustration and example only and not limitation. The scope of the present invention should be interpreted by terms of the appended claims.

What is claimed is:

1. A medical information processing device comprising:
a hardware processor that:
acquires data regarding a human body;
accumulates acquired data acquired by the hardware processor;
analyzes accumulated data accumulated by the hardware processor, autonomously finds a correspondence pattern between the accumulated data and at least timing at which prescription is performed and a content out of the accumulated data and generates an identifier used for inference;
infers a prescription content including at least the timing at which the prescription should be performed using the identifier generated by the hardware processor and the acquired data; and
outputs the prescription content inferred by the hardware processor.

2. The medical information processing device according to claim 1, wherein the hardware processor infers a type of the prescription together as the prescription content.

3. The medical information processing device according to claim 1, wherein the hardware processor is a display device capable of displaying the prescription content inferred by the hardware processor.

4. The medical information processing device according to claim 1, comprising: a reason presenting part that presents a reason that the hardware processor infers an inference content output by the hardware processor.

5. The medical information processing device according to claim 1, comprising:
an operator operable by a user,
wherein the hardware processor is capable of making an input based on operation performed on the operator the acquired data.

6. The medical information processing device according to claim 1,
wherein the hardware processor is capable of generating a plurality of types of identifiers by making the accumulated data used in machine learning different, and
the hardware processor is capable of inferring a plurality of types of prescription contents by using the plurality of types of identifiers and the acquired data.

7. The medical information processing device according to claim 1, wherein the acquired data includes at least image data and text data.

8. The medical information processing device according to claim 7, wherein the image data includes at least two types of image data acquired from different modalities.

9. The medical information processing device according to claim 1, wherein the type of the prescription includes an examination condition.

10. The medical information processing device according to claim 1, wherein the hardware processor performs deep learning as the machine learning.

11. A non-transitory recording medium storing a computer readable program causing a computer to perform:
acquiring data regarding a human body;
accumulating acquired data acquired by the hardware processor;
performing machine learning to generate an identifier used for inference by learning a correspondence pattern between accumulated data and at least timing at which prescription is performed and a content based on the accumulated data accumulated by the hardware processor; and
inferring a prescription content including at least the timing at which the prescription should be performed using the identifier generated by the hardware processor and the acquired data.
